(19) United States
(12) Patent Application Publication
(10) Pub. No.: US 2017/0107553 A1
(43) Pub. Date: Apr. 20, 2017

(54) CELL CULTURE PROCESS FOR PRODUCING A PROTEIN

(71) Applicant: Lupin Limited, Mumbai (IN)

(72) Inventors: Sridhar Kottakota, Pune (IN); Sorab Rustom Mody, Pune (IN)

(21) Appl. No.: 15/315,778

(22) PCT Filed: Jun. 3, 2015
(86) PCT No.: PCT/IB2015/054196

§ 371 Date: Dec. 2, 2016

(30) Foreign Application Priority Data
Jun. 3, 2014 (IN) 1827/MUM/2014

(51) Int. Cl. C12P 21/02; C12N 5/071; C07K 16/32; C07K 16/22; C07K 16/28; C07K 16/24; C12P 21/00; C07K 14/705

(52) U.S. Cl. CPC C12P 21/02; C12P 21/005; C12N 5/0682; C07K 14/70578; C07K 16/22; C07K 16/2887; C07K 16/241; C07K 16/32; C12N 2500/90; C07K 2317/14; C07K 2317/41; C07K 2319/30; C07K 2317/24

(57) ABSTRACT

The invention provides the mammalian cell culture process for the production of monoclonal antibodies and fusion proteins wherein the mammalian cell is cultured in suitable cell culture conditions specifically maintaining monophasic temperature.

### FIELD OF THE INVENTION

The invention is related to the monophasic mammalian cell culture conditions for the production of glycosylated proteins. More particularly, it relates to the production of monoclonal antibodies and fusion proteins in mammalian cell culture conditions having monophasic temperature condition. More particularly, the invention is related to process for culturing mammalian cell in suitable cell culture conditions for the production of proteins preferably glycosylated proteins. More particularly, it also relates to the process of cell culture performing with suitable cell culture conditions specifically maintaining monophasic temperature for the production of glycosylated proteins

### BACKGROUND OF THE INVENTION

The production of therapeutic proteins for biopharmaceutical applications typically involves the use of mammalian cell cultures that are known to produce high level of glycosylated proteins. Control and optimization of mammalian cell culture conditions is critically important for successful commercial production of glycosylated proteins. Conventionally the mammalian cell culture process for many of the glycosylated proteins is biphasic; distinguished by an initial growth phase and subsequent production phase. This dual production phases provides high cell densities and better product titer.

Furthermore, controlling and optimizing cell culture conditions have always been a great challenge because these conditions drastically affect the cell viability, desired product yield, purity and heterogeneity. The physio-chemical and pharmacokinetics properties of the therapeutic protein molecules critically depend over the culture conditions.

U.S. Pat. No. 7,294,481 discloses a method for producing a fusion protein, i.e., TNFR:Fc. During the production phase, the host cells were cultured at a temperature of 25-34° C., which showed reduction in disulfide scrambling in the TNFR:Fc produced in comparison to the production phase carried out at 37° C. Further it discloses that the production phase is carried out in the presence of an alkanoic acid or salt thereof.

The initiation of the production phase may be achieved in numerous ways, with temperature and pH shifts being the most common. Other methods used are addition of inducing agents, alteration of feed substrate(s) or osmolality changes.

The invention relates to the cell culture process by maintaining suitable culture condition during the culture. Specifically, the mammalian cells are grown at a single temperature selected from at about 34° C. to at about 37° C. The invention relates to the cell culture process in fed batch mode by maintaining suitable culture condition during the culture.

### SUMMARY OF THE INVENTION

In an embodiment, the invention is related to monophasic mammalian cell culture process to produce glycosylated proteins.

In another embodiment, the invention is related to a monophasic temperature mammalian cell culture process to produce glycosylated proteins.

In another embodiment, the invention is related to a monophasic temperature mammalian cell culture process to produce glycosylated proteins, wherein the temperature is set between 34° C. to 37° C.

In another embodiment, the invention is related to mammalian cell culture process having monophasic temperature condition to produce fusion proteins and monoclonal antibodies.

In yet another embodiment, the invention is related to the method of producing glycosylated proteins at a monophasic temperature for mammalian cell culture wherein the cell culture is essentially free of alkanoic acid or salt thereof.

In another embodiment, the invention is related a process of producing glycosylated protein in a mammalian cell culture the process comprising the steps of:
a) preparing inoculum with suitable cell concentration during seed development;
b) inoculating the inoculum with suitable cell concentration in to production bioreactor;
c) culturing the cell in production bioreactor at suitable conditions wherein the suitable condition is monophasic temperature condition; and
d) obtaining the glycosylated protein from the cell culture.

In another embodiment, the invention is related a process of producing glycosylated protein in a mammalian cell culture the process comprising the steps of:
a) preparing inoculum with suitable cell concentration during seed development;
b) inoculating the inoculum with suitable cell concentration in to production bioreactor;
c) culturing the cell in production bioreactor at suitable conditions wherein the suitable condition is monophasic temperature condition; and
d) obtaining the glycosylated protein from the cell culture,
   wherein monophasic temperature does not have temperature shift during step (c).

In another embodiment, the invention is related a process of producing glycosylated protein in a mammalian cell culture the process comprising the steps of:
a) preparing inoculum with suitable cell concentration during seed development;
b) inoculating the inoculum with suitable cell concentration in to production bioreactor;
c) culturing the cell in production bioreactor at suitable conditions wherein the suitable condition is monophasic temperature condition; and
d) obtaining the glycosylated protein from the cell culture,
   wherein the production bioreactor in step (c) does not have any distinctive growth phase and production phase.

In another embodiment, the invention is related to cell culture process performing with monophasic temperature condition to produce glycosylated proteins, wherein the temperature is maintained at a set point between at about 32° C. to about 37° C.

In another embodiment, the invention is related to cell culture process performing with monophasic temperature condition to produce glycosylated proteins, wherein the temperature is maintained at a set point between at about 34° C. to about 37° C.

In certain embodiment, the invention is related to cell culture process performing with monophasic temperature condition to produce glycosylated proteins, wherein the temperature is maintained at about 33° C.

In certain embodiment, the invention is related to cell culture process performing with monophasic temperature condition to produce glycosylated proteins, wherein the temperature is maintained at about 34° C.

In certain embodiment, the invention is related to cell culture process performing with monophasic temperature condition to produce glycosylated proteins, wherein the temperature is maintained at about 35° C.

In certain embodiment, the invention is related to cell culture process performing with monophasic temperature condition to produce glycosylated proteins, wherein the temperature is maintained at about 36° C.

The fusion proteins and monoclonal antibodies produced by the processes of the invention are useful for biopharmaceutical applications.

The details of one or more embodiments of the invention set forth below are illustrative in nature only and not intended to limit to the scope of the invention. Other features, objects and advantages of the inventions will be apparent from the description.

### BRIEF DESCRIPTION OF FIGURES

FIG. 1 depicts comparative growth trends of CHO cell observed during the Etanercept production.

FIG. 2 depicts comparative viability trends of CHO cell observed during the Etanercept production.

FIG. 3 depicts comparative growth trends of CHO cell observed during the Bevacizumab production.

FIG. 4 depicts comparative viability trends of CHO cell at various time points observed during Bevacizumab production.

FIG. 5 depicts growth trends of CHO cell in Rituximab production

FIG. 6 depicts viability trends of CHO cell in Rituximab production

FIG. 7 depicts growth trends of CHO cell in Trastuzumab production

FIG. 8 depicts viability trends of CHO cell in Trastuzumab production

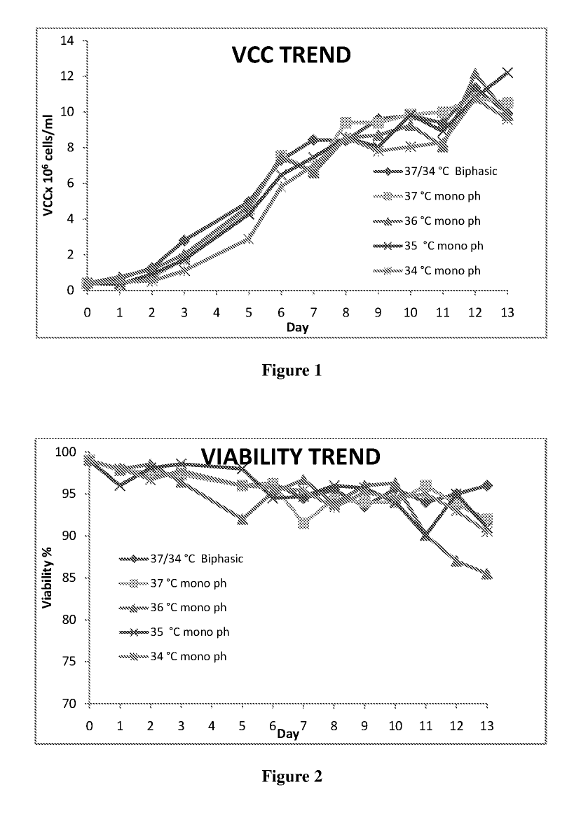

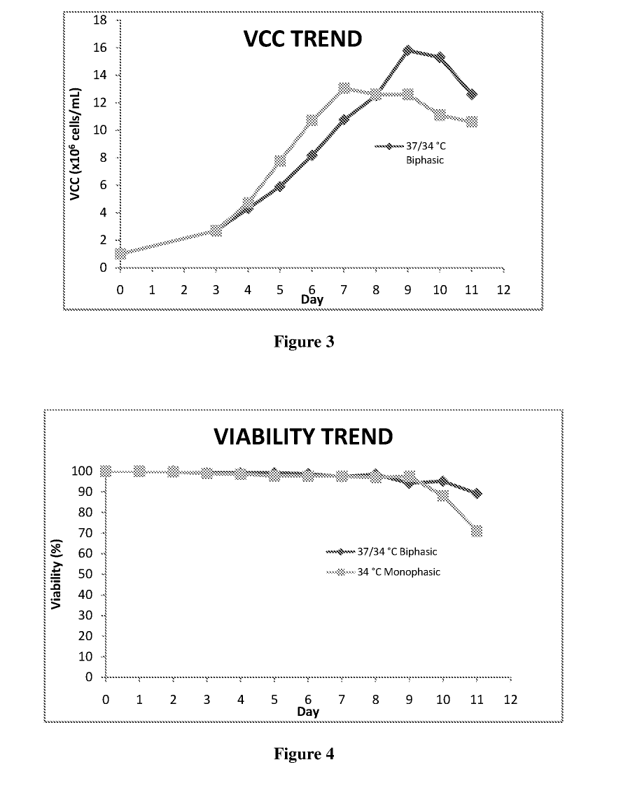

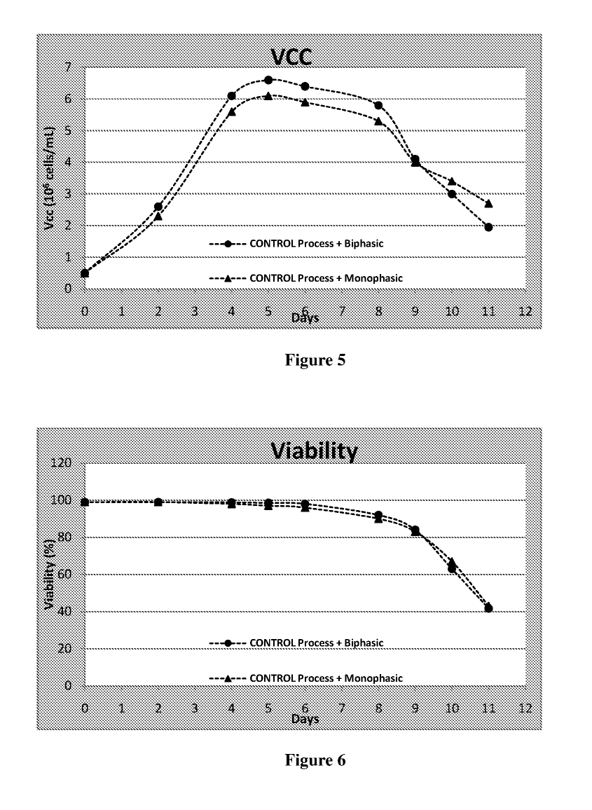

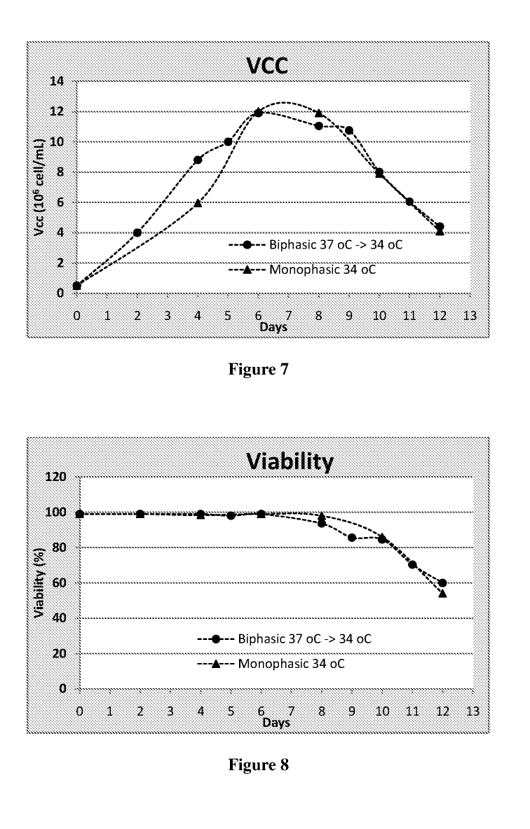

### DETAILED DESCRIPTION OF THE INVENTION

Definition:

The term “antibody” as referred to herein includes whole antibodies and any antigen binding fragments or single chains thereof. An “antibody” refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions may be further subdivided into regions of hypervariability, termed complementarity determining regions (CDR) with are hypervariable in sequence and/or involved in antigen recognition and/or usually form structurally defined loops, interspersed with regions that are more conserved, termed framework regions (FR or FW). Each VH and VL is composed of three CDRs and four FWs, arranged from amino-terminus to carboxy-terminus in the following order: FW1, CDR1, FW2, CDR2, FW3, CDR3, FW4. The amino acid sequences of FW1, FW2, FW3, and FW4 all together constitute the “non-CDR region” or “non-extended CDR region” of VH or VL as referred to herein.

The ‘Host cell” is genetically engineered means have recombinant DNA or RNA to expresses a gene at elevated levels or at lowered levels, or expresses a mutant form of the gene. In other words, the cell has been transfected, transformed or transduced with a recombinant polynucleotide molecule, and thereby altered so as to cause the cell to alter expression of a desired polypeptide. The conventional methods of “genetic engineering” are known in the prior art.

“Production medium” means a cell culture medium designed to be used to culture cells during a production phase.

As used herein, “monophasic” refers to cell culture method which involves no change in any of the culture conditions at which the culture is maintained. The culture conditions includes but are not limited to temperature, pH, osmolality or chemical excipients.

As used herein, “monophasic temperature” refers to cell culture method performed with the use of a single temperature set point in the production bioreactor (referred to as passage “N”). And the temperature is maintained at a single set point during the run of production bioreactor to obtain monophasic growth conditions. The monophasic is restrained to shift. The monophasic temperature is between at about 34° C. to at about 37° C.

As used herein, “glycosylated protein” refers to one or more mammalian polypeptides that function as a discrete unit. The “glycosylated protein” includes fusion proteins and monoclonal antibodies used for biopharmaceutical applications. Examples of fusion protein include but are not limited to etanercept, abatacept, alefacept, rilonacept, belatacept, aflibercept, etc. Examples of monoclonal antibodies include but are not limited to rituximab, trastuzumab, bevacizumab, adalimumab, denosumab, palivizumab, cetuximab, omalizumab, natalizumab, panitumumab, ustekinumab, ofatumumab, pertuzumab, etc.

As used herein, “Cell density” refers to that number of cells present in a given volume of medium.

As used herein, “mammalian cell culture” refers to a cell population that is suspended in a medium under conditions suitable to survival and/or growth of the cell population. It refers to growth and propagation of mammalian cells outside of a multicellular organism or tissue. Suitable culture conditions for mammalian cells are known in the art. Mammalian cells may be cultured in suspension or while attached to a solid substrate.

As used herein, “fed-batch culture” refers to a method of culturing cells in which additional components are provided to the culture at some time subsequent to the beginning of the culture process. The provided components typically comprise nutritional supplements for the cells which have been depleted during the culturing process.

As used herein, mammalian cell culture process refers to the use of recombinant mammalian cell lines such as CHO DUKX-B11, CHO S, CHO K1, CHO DG44.

As used herein, the starting VCC (viable cell count) after inoculation of seed in the monophasic Production Bioreactor, are selected from 0.1×10⁶ cells/mL to 10×10⁶ cells/mL and viability >90%, more preferably at 0.3×10⁶ cells/mL to 5×10⁶ cells/mL and viability >90% and even more preferably at 1×10⁶ cells/mL to 2×10⁶ cells/mL and viability >90%. The VCC may be measured during the process at suitable time intervals.

As used herein, “about” with reference to temperature refers to deviation in temperature value wherein it covers ±1° C. e.g. about 33° C. covers temperature from 32° C. to 34° C.

As used herein, “about” with reference to pH refers to deviation in pH value wherein it covers ±0.5 e.g. about pH 6.7 covers pH from 6.2 to 7.2.

In another embodiment, the invention is related to process for culturing mammalian cell carried out with monophasic temperature condition to produce fusion proteins and monoclonal antibodies.

In yet another embodiment, the invention is related to the mammalian cell culture process performed with monophasic temperature condition to produce glycosylated proteins wherein the cell culture is essentially free of alkanoic acid or salt thereof.

In another embodiment the invention is related to cell culture process performed with monophasic temperature condition at about 34° C. to about 37° C. to produce glycosylated proteins which improve the desired confirmation of glycosylated protein.

In another embodiment the mammalian cell culture is carried out in batch, fed-batch and continuous mode in suitable medium in fermenter or bioreactor, preferably in fed-batch mode. The invention is related to the process of culturing the mammalian cell for the production of glycosylated proteins. More particularly, the invention relates to specific culture conditions which are maintained during the mammalian cell culture process.

Although a lot of processes have been developed and reported in the literature for the production of glycosylated protein from the mammalian cell culture, however those processes are typically biphasic, requiring two sets of temperatures and/or dissolved oxygen and/or pH. In the biphasic cell culture condition the recombinant host cells are first grown at a temperature which promotes rapid cell multiplication, referred to as the growth phase. This is typically achieved by growing cells at 34-37° C., then lowering the temperature to 22-34° C. which reduces cell growth while favoring the production of the recombinant protein, referred to as the production phase.

The cell culture process requires various parameters to carry out the process in effective and efficient way. However, the main aspect of the invention is the use of monophasic temperature during the culture wherein the monophasic temperature does not have temperature shift and produce desire quality and quantity of the glycosylated protein with significant cell viability.

The invention studies the effect of temperature over the production of protein, its quality and cell viability by using the techniques known in the art. Temperature is selected from 37° C. or 36° C. or 35° C. or 34° C. or 33° C.

In another embodiment monophasic temperature condition at about 34° C. to about 37° C. improve the desired confirmation of glycosylated protein.

In another embodiment the invention is related to cell culture process for the production of glycosylated proteins in monophasic cell culture condition wherein the mammalian cells are cultured at temperature maintained between from at about 32° C. to at about 37° C.

In another embodiment the invention is related to cell culture process for the production of glycosylated proteins in monophasic cell culture condition wherein the mammalian cells are cultured at temperature maintained between from at about 34° C. to at about 37° C.

In one embodiment of the invention, the monophasic temperature is set at about 33° C.

In one embodiment of the invention, the monophasic temperature is set at about 34° C.

In one embodiment of the invention, the monophasic temperature is set at about 35° C.

In one embodiment of the invention, the monophasic temperature is set at about 36° C.

In another embodiment of the invention, the monophasic temperature is set at about 37° C.

In an embodiment, the invention is related a process of producing glycosylated protein in a mammalian cell culture the process comprising the steps of:
a) preparing inoculum with suitable cell concentration during seed development;
b) inoculating the inoculum with suitable cell concentration in to production bioreactor;
c) culturing the cell in production bioreactor at suitable conditions wherein the suitable condition is monophasic temperature condition; and
d) obtaining the glycosylated protein from the cell culture.

In an embodiment, the invention is related a process of producing glycosylated protein in a mammalian cell culture the process comprising the steps of:
a) preparing inoculum with suitable cell concentration during seed development;
b) inoculating the inoculum with suitable cell concentration in to production bioreactor;
c) culturing the cell in production bioreactor at suitable conditions wherein the suitable condition is monophasic temperature condition; and
d) obtaining the glycosylated protein from the cell culture, wherein monophasic temperature does not have temperature shift during step (c).

In an embodiment, the invention is related a process of producing glycosylated protein in a mammalian cell culture the process comprising the steps of:
a) preparing inoculum with suitable cell concentration during seed development;
b) inoculating the inoculum with suitable cell concentration in to production bioreactor;
c) culturing the cell in production bioreactor at suitable conditions wherein the suitable condition is monophasic temperature condition; and
d) obtaining the glycosylated protein from the cell culture,
   wherein the production bioreactor in step (c) does not have any distinctive growth phase and production phase.

In another embodiment the seed development steps carried out to develop the inoculum having suitable cell concentration. It required at least about 72 hours to develop inoculum with desired cell concentration thereafter the inoculum is inoculated in to the production bioreactor for further scale up and protein production.

In another embodiment the cell culture process is carried out in production fermenter or production bioreactor. The cells may be cultured for total period of 9 to 40 days. In another embodiment, the protein is harvested at least before the day 15, preferably on day 13, more preferably on day 12 and most preferably on day 11. In another embodiment the protein is harvested before the cell viability reached below ≦90%. In another embodiment the protein is harvested before the cell viability reached below ≦70%. In another embodiment the protein is harvested before the cell viability reached below ≦50%. In preferred embodiment the suitable culture condition is monophasic temperature which does not have temperature shift. Temperature is selected from about 33° C. to about 37° C. preferably from about 33° C. to about 36° C.

In another embodiment the suitable conditions during the culture may be further selected from pH, osmolality, dissolved oxygen concentration and cell density.

The seed preparation is initiated with suitable concentration of cells which are selected from 0.1×10⁶ cells/mL to 0.5×10⁶ cells/mL in suitable medium. In preferred embodiment seed preparation is initiated with 0.3×10⁶ cells/mL in suitable medium. In another preferred embodiment seed preparation is initiated with 0.3×10⁶ cells/mL in suitable medium and further supplemented with suitable concentration of glutamine and methotrexate. The suitable concentration of glutamine and methotrexate is selected from 3 mM to 6 mM, preferably 4 mM and 70 nM to 100 nM, preferably 80 nM respectively. In another embodiment the suitable concentration of glutamine is 6 mM.

In certain embodiment the seed culture is maintained at about 3% to 8% CO₂, preferably 5% CO₂, dissolved oxygen concentration is selected from at about 30% to about 70% preferably at 50%, relative humidity is selected from about 70% to about 90%, preferably 85%, agitation speed is selected from about 0.2 m/s to about 0.4 m/s, preferably 0.3 m/s and temperature is selected from between at about 34° C. to at about 37° C., preferably 37° C. In certain embodiment the concentration of dissolved oxygen concentration is maintained by sparging with air at 0.00 to 0.03 vvm and oxygen at 0.00 to 0.09 vvm. In certain embodiment the seed culture is further amplified by passage by diluting to at least 0.1×10⁶ cells/mL. The dilution may be performed during intervals of seed culture. In another embodiment the dilution is performed at least on 3^{rd} day or at 72 hours.

In preferred embodiment the seed culture is further amplified by supplementing feed till the culture grow to suitable concentration of cell. The seed culture is further amplified by supplementing feed till the culture grows to suitable concentration of cell for suitable time period. The seed culture may be further amplified by supplementing feed for at least 5 days and the suitable concentration of cell i.e. inoculum is selected from about 4×10⁶ cells/mL to about 7×10⁶ cells/mL preferably 5×10⁶ cells/mL.

In embodiment the feed is supplemented to the cell culturing in suitable bioreactor or fermenter in suitable culture conditions. In certain embodiment the cells are cultured at suitable culture conditions is selected from about 3% to 6% CO₂, preferably 5% CO₂, dissolved oxygen concentration is selected from at about 30% to about 70% preferably at 50%, relative humidity is selected from about 70% to about 90%, preferably 85%, agitation speed is selected from about 0.2 m/s to about 0.4 m/s, preferably 0.3 m/s and temperature is selected from between at about 34° C. to at about 37° C., preferably 37° C. In certain embodiment the concentration of dissolved oxygen concentration is maintained by sparging with air at 0.03 vvm to 0.09 vvm.

In embodiment the viability is cell during the seed culture is maintained at least by >70%, preferably >80%, more preferably >90%.

In preferred embodiment the cells were taken from a cell bank and cultured in a shake flask containing growth medium with or without methotrexate to an initial viable cell density (VCC) of 0.3×10⁶ cells/mL. The seed culture flask was then maintained at a set temperature of 37° C. at 5% CO₂ concentration at 120 rpm and approx. 85% relative humidity. The seed volume was volumetrically amplified during subsequent passages by diluting to 0.3×10⁶ cells/mL after every 72 hr. The N-1 seed was additionally supplemented with 10% of feed on day 1 and day 3 in bioreactor (or flasks) and incubated for 120 hr. Log phase cells from the N-2 bioreactor (or flasks) at a VCC of 2 to 2.5×10⁶ cells/mL and viability >90% was used to inoculate the N-1 bioreactor (or flasks). The N-1 bioreactor was maintained at a tip speed of ˜0.3 m/s with dissolved oxygen at approx. 50% of dissolved oxygen saturation by sparging separately air at 0.00 to 0.03 vvm and oxygen at 0.00 to 0.09 vvm, respectively to achieve a final VCC of 2 to 10×10⁶ cells/mL and viability >90%, more preferably at 4 to 8×10⁶ cells/mL and viability >90% and even more preferably at 5 to 6×10⁶ cells/mL and viability >90%.

The suitable concentration of cells obtained from seed culture is referred as inoculum which is transferred to bioreactor or fermenter to initiate the culture at high density. In certain embodiment the suitable concentration of inoculum is selected from about 4×10⁶ cells/mL to about 7×10⁶ cells/mL preferably 5×10⁶ cells/mL

The batch or the production bioreactor is initiated with inoculum having suitable concentration of cells which is selected from 4×10⁶ cells/mL to 7×10⁶ cells/mL in suitable medium. In preferred embodiment seed density is 1.2×10⁶ cells/mL (which may be achieved by diluting 5×10⁶ cells/mL cells/mL or 6×10⁶ cells/mL cells/mL obtained through seed preparation). In another preferred embodiment seed density is 1×10⁶ cells/mL. In certain preferred embodiment the cells are culture in suitable medium and further supplemented with suitable concentration of glutamine The suitable concentration of glutamine is selected from 3 mM to 6 mM, preferably 4 mM.

In certain embodiment the production bioreactor is maintained at about pH from about 6 to about 8, preferably about 6.7 to 7.4, more preferably 7, 3% to 6% CO₂, preferably 5% CO₂, dissolved oxygen concentration is selected from at about 30% to about 70% preferably at 50%, agitation speed is selected from about 0.2 m/s to about 0.5 m/s, preferably 0.3 m/s and temperature is selected from between at about 34° C. to at about 37° C., preferably 34° C. In certain embodiment the concentration of dissolved oxygen concentration is maintained by sparging with air at 0.03 vvm to 0.09 vvm. The sodium bicarbonate and CO₂gas is used to control the pH of the culture.

In embodiment the feeding is performed during the batch/bioreactor culture based on the residual glucose level. The glucose level is adjusted to at least 2 g/L, if the glucose level is below 2 g/L, feed is supplemented to maintain the suitable glucose level. The glucose concentration of the culture is monitored in every 12 hr or 18 hr or 24 hr.

In embodiment the feed may contain from about 30 to about 35 g/L of glucose.

In another embodiment the feed may contain from about 180 to about 220 mM, L-glutamine, preferably 200 mM of L-glutamine solution. In one embodiment the L-glutamine solution is added at 1% of the initiation volume to the culture at 2 days and then subsequently in every 2 day still 11 days. In another embodiment the L-glutamine solution is added at 1% of the initiation volume to the culture at 4 days and then at 7 days and at 9 days.

In another embodiment the D-glucose is supplemented as feed to the culture. The concentration of D-glucose is selected from about 60 g to about 90 g, preferably 80 g. In certain embodiment the D-glucose is supplemented as feed to the culture at least after 9 days or at least from 10 days.

In another embodiment the desire protein is harvested at least after 10 days or after 11 days or after 12 days or after 13 days.

In another embodiment the harvesting of the culture is performed when culture viability drops below 40% to 70%, preferably below 50%.

In another embodiment the production bioreactor process was initiated by inoculating N-1 seed in to a bioreactor containing growth medium at approx. 55% of the final batch volume at the starting VCC (after inoculation of the seed) in the monophasic Production Bioreactor of 0.1 to 10×10⁶ cells/mL and viability >90%, more preferably VCC at 0.3-5×10⁶ cells/mL and viability >90% and even more preferably VCC at 1 to 2×10⁶ cells/mL and viability >90%. The culture pH was maintained at pH ranging between 6.7 to 7.4 by addition of 8% sodium bicarbonate (NaHCO₃) or CO₂ gas. The agitation speed was set as per the tip speed ranging from 0.3 to 0.5 m/s, and dissolved oxygen concentration maintained at 50% dissolved oxygen saturation controlled by sparging air at 0.00 to 0.03 vvm and oxygen at 0.00 to 0.09 vvm. The temperature was set at a single set point between 34° C. to 37° C., preferably 34° C. throughout the Production Bioreactor process (monophasic). Feeding of the reactor was done based on the residual glucose levels. The glucose concentration of the culture monitored every 24 hr. and adjusted to 3 g/L if the glucose level is below 2 g/L with addition of the feed. The feed contains 33.5 g/l glucose. 200 mM of L-glutamine solution was added at 1% of the initiation volume to the culture at 48 hr. and every 48 hr. thereon up to 240 hr. Harvesting of the culture was done on the 264 hr. or if the culture viability drops below 90%, whichever is attained first.

The cell culture process was initiated in bioreactor with the viable cell concentration which is selected from about 1×10⁶ cells/mL to 2×10⁶ cells/mL, preferably 1.2×10⁶ cells/mL. In another embodiment the viable cell concentration is 1×10⁶ cells/mL. In certain embodiment, the suitable conditions for culturing the cell is selected from pH 6.7 to 7.4, preferably 7.0. In another embodiment the osmolality is selected from about 250 to about 550 mOSm/Kg. In another embodiment the dissolved oxygen is selected from at about 30% to about 70% preferably at 50% set point.

In preferred embodiment the growth and production phase is not distinctive in the production bioreactor.

In another embodiment the production phase may batch or fed-batch.

In embodiment the harvested protein is clarified by the techniques known in art to skilled person. In preferred embodiment the harvested protein present in broth obtained from bioreactor or fermenter. EDTA solution is added in suitable concentration which could be 5 mM and then clarification carried out depth filtration using POD system.

The media composition is very important to improve the culture longevity and production. Basal cell culture medium formulations are well known in the art. To these basal culture medium formulations the skilled artisan will add components such as amino acids, salts, sugars, vitamins, hormones, growth factors, buffers, antibiotics, lipids, trace elements and the like, depending on the requirements of the host cells to be cultured. The culture medium may or may not contain serum and/or protein. Various tissue culture media, including serum-free and/or defined culture media, are commercially available for cell culture. Tissue culture media is defined, for purposes of the invention, as a media suitable for growth of animal cells, and preferably mammalian cells, in in vitro cell culture. Typically, tissue culture media contains a buffer, salts, energy source, amino acids, vitamins and trace essential elements. Any media capable of supporting growth of the appropriate eukaryotic cell in culture is used;

the invention is broadly applicable to eukaryotic cells in culture, particularly mammalian cells, and the choice of media is not crucial to the invention. Tissue culture media suitable for use in the invention are commercially available from, e.g., ATCC (Manassas, Va.). For example, any one or combination of the following media is used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EXCELL™ 300 Series. In preferred embodiment the medium is EX-CELL™ 302 medium.

In another preferred embodiment the mix feed media comprises BalanCD CHO Feed 2, EX-CELL 302 Powder Medium, NaHCO3, L-Glutamine Powder, MEM Amino Acid (50 X), MEM Non-Essential Amino Acid (100 X), MEM Vitamins Solution (100 X).

In the methods and compositions of the invention, cells may be grown in serum-free, protein-free, growth factor-free, and/or peptone-free media. The term “serum-free” as applied to media includes any mammalian cell culture medium that does not contain serum, such as fetal bovine serum.

The skilled artisan may also choose to use one of the many individualized media formulations that have been developed to maximize cell growth, cell viability, and/or recombinant polypeptide production in a particular cultured host cell. The methods according to the current invention may be used in combination with commercially available cell culture media or with a cell culture medium that has been individually formulated for use with a particular cell line

In preferred embodiment the medium is serum free. In another preferred embodiment the medium is essentially free of alkanoic acid or salt thereof. The alkanoic acid and salt thereof are selected from butyric acid, sodium butyrate or dibutyl cAMP.

In embodiment glycosylated proteins are selected from Abciximab; Abatacept; Adalimumab; Abrilumab; Afutuzumab; Aflibercept; Alemtuzumab; Alefacept; Alacizumab pegol; Anakinra; Arcitumomab; Atacicept; Atlizumab; Atorolimumab; Basiliximab; Baminercept; Bectumomab; Belimumab; Besilesomab; Bevacizumab; Biciromab; Belatacept; Brentuximab vedotin; Brodalumab; Canakinumab; Capromab pendetide; Catumaxomab; Certolizumab pegol; Cetuximab; Clivatuzumab tetraxetan; Daclizumab; Denosumab; Eculizumab; Edrecolomab; Efalizumab; Efungumab; Eloctate; Ertumaxomab; Etanercept; Etaracizumab; Fanolesomab; Farletuzumab; Fontolizumab; Gemtuzumab ozogamicin; Girentuximab; Golimumab; Ibritumomab tiuxetan; Igovomab; Imciromab; Infliximab; Ipilimumab; Labetuzumab; Mepolizumab; Motavizumab; Muromonab-CD3; Natalizumab; Nimotuzumab; Nofetumomab merpentan; Obinutuzumab; Ofatumumab; Omalizumab; Oregovomab; Palivizumab; Panitumumab; Pemtumomab; Pertuzumab; Ramucirumab; Ranibizumab; Raxibacumab; Rituximab; Rilonacept; Rovelizumab; Ruplizumab; Sulesomab; Tacatuzumab tetraxetan; Tefibazumab; Tocilizumab; Trastuzumab; Ado-Trastuzumab Emtansine; Tositumomab; TRBS07; Ustekinumab; Vedolizumab; Visilizumab; Votumumab; Zalutumumab; Zanolimumab.

The purification of the polypeptide may include an affinity column containing agents which will bind to the polypeptide; one or more column steps over such affinity resins as concanavalin A-agarose, heparin-TOYOPEARL® (Togo Soda Manufacturing Co., Ltd., Japan) or Cibacrom blue 3GA SEPHAROSE® (Pharmacia Fine Chemicals, Inc., New York); one or more steps involving elution; and/or immunoaffinity chromatography. The polypeptide may be expressed in a form that facilitates purification. For example, it may be expressed as a fusion polypeptide, such as those of maltose binding polypeptide (MBP), glutathione-S-transferase (GST), or thioredoxin (TRX). Kits for expression and purification of such fusion polypeptides are commercially available from New England BioLab (Beverly, Mass.), Pharmacia (Piscataway, NI) and InVitrogen, respectively. The polypeptide may be tagged with an epitope and subsequently purified by using a specific antibody directed to such epitope. One such epitope (FLAG®) is commercially available from Kodak (New Haven, Conn.). It is also possible to utilize an affinity column comprising a polypeptide-binding protein, such as a monoclonal antibody to the recombinant polypeptide, to affinity-purify expressed polypeptides. Other types of affinity purification steps may be a Protein A or a Protein G column, which affinity agents bind to proteins that contain Fc domains. Polypeptides may be removed from an affinity column using conventional techniques, e.g. in a high salt elution buffer and then dialyzed into a lower salt buffer for use or by changing pH or other components depending on the affinity matrix utilized, or may be competitively removed using the naturally occurring substrate of the affinity moiety.

Although certain embodiments in detail above and the invention may be illustrated by way of examples below, those having ordinary skill in the art will clearly understand that many modifications are possible in the embodiments and examples without departing from the teachings thereof

### EXAMPLES

The CHO cell line was established by co-transfection of the dihydrofolate reductase (dhfr) and gene of interest transfected into dhfr-deficient CHO cells (DUKX-B 11, ATCC CRL-9096) followed by subsequent dhfr/MTX-mediated gene amplification. The clones were prepared as per the techniques well known in the art related to recombinant r-DNA technology.

### Example 1

### Production of Etanercept through Monophasic Process

Seed Expansion Process

The seed expansion was initiated post revival of a vial from the cell bank at 0.3×10⁶ cells/mL in growth medium supplemented with 4 mM L-Glutamine with MTX. The concentration of MTX was maintained at 80 nM throughout the stage of seed development. The seed culture was maintained at 37° C. at 5% CO2 concentration at 120 rpm and approx. 80% Relative humidity in shake flask.

The seed volume was adequately amplified at every subsequent passage by diluting to 0.3×10⁶ cells/mL after every 72 hr. at log phase. The N-2 seed was initiated with 0.3×10⁶ cells/mL and additionally supplemented with 10% of mixed feed (of the initiation volume) on 24 hr and 72 hr and cultured for 120 hr.

Log phase cells from the N-2 flask at a VCC of 5.5×10⁶ cells/mL and viability >95% was used to inoculate the N-1 flask with an initial seed density of 1.2×10⁶ cells/mL.

The N-1 seed flask was run for 120 hr with feeding with 10% mixed feed (of the initiation volume) at 24 hr and 72 hr to obtain a viable cell density of 6.6×10⁶ cells/mL and a viability above >95%.

Production Bioreactor Process

The batch process was initiated by inoculating N-1 seed in to a bioreactor containing growth medium at approx. 55% of the final batch volume at the starting VCC (after inoculation of the seed) in the Monophasic Production Bioreactor of 1.2×10⁶ cells/mL and viability >98%. The culture pH was maintained at pH ranging between 6.7 to 7.4 by addition of 8% sodium bicarbonate (NaHCO₃) or CO₂ gas. The agitation speed was set as per the tip speed ranging from 0.3 to 0.5 m/s, and dissolved oxygen concentration maintained at 50% dissolved oxygen saturation by sparging air at 0.00 to 0.03 vvm and oxygen at 0.00 to 0.09 vvm. The temperature was set at a single set point between 34° C. throughout the Production Bioreactor process (monophasic). Feeding of the reactor was done based on the residual glucose levels. The glucose concentration of the culture monitored every 24 hr. and adjusted to 3 g/L every 24 hr if the residual glucose level is below 2 g/L with addition of the feed (the feed contains 33.5 g/l glucose). 200 mM of L-glutamine solution was added at 1% of the initiation volume to the culture at 48 hr. and every 48 hr thereon up to 240 hr. The culture attained a peak VCC of around 11×10⁶ cells/mL. Harvesting of the culture was done on the 264 hr.

### Example 2

### Production of Bevacizumab through Monophasic Process

Seed Expansion Process

The seed expansion was initiated post revival of a vial from the cell bank at 0.4×10⁶ cells/mL in growth medium supplemented with 6 mM L-Glutamine. The seed culture was maintained at 37° C. at 8% CO₂ concentration at 150 rpm and approx. 80% Relative humidity in shake flask.

The seed volume was adequately amplified at every subsequent passage by diluting to 0.4×10⁶ cells/mL after every 72 hr. at log phase.

The N-1 seed flask was run for 72 hr to obtain a viable cell density of 5.5×10⁶ cells/mL and a viability above >95%.

Production Bioreactor Process

The batch process was initiated by inoculating N-1 seed in to a bioreactor containing growth medium at approx. 69% of the final batch volume at the starting VCC (after inoculation of the seed) in the Monophasic Production Bioreactor of 1×10⁶ cells/mL and viability >98%. The culture pH was maintained at pH ranging between 6.8 to 7.4 by addition of 8% sodium bicarbonate (NaHCO3) or CO2 gas. The agitation speed was set as per the tip speed ranging from 0.3 to 0.5 m/s, and dissolved oxygen concentration maintained at 50% dissolved oxygen saturation controlled by sparging air at 0.00 to 0.03 vvm and oxygen at 0.00 to 0.09 vvm. The temperature was set at a single set point between 34° C. throughout the Production Bioreactor process (monophasic). Feeding of the reactor was done based on the residual glucose levels. The glucose concentration of the culture monitored every 24 hr. and adjusted to 4 g/L every 24 hr if the residual glucose level is below 2 g/L with addition of the feed (the feed contains 33.5 g/l glucose). 200 mM of L-glutamine solution was added at 1% of the initiation volume to the culture on 96, 168 and 216 hr. The culture attained a peak VCC of around 11×10⁶ cells/mL Harvesting of the culture was done on the 264 hr.

### Example 3

### Production of Rituximab through Monophasic Process

Seed Expansion Process

A vial of rituximab from liquid nitrogen was thawed and the cells were inoculated in a 125 mL shake flask containing growth medium and was cultured in CO₂ incubator shaker at 37° C., 120 rpm. Cells were passaged every 72 h±24 h with increase in culture volume appropriate for inoculating the production bioreactor. In each passage seeding density was maintained at 0.3×10⁶ cells/mL and target for a final VCC of 3.0±1.0×10⁶ cells/mL.

Production Bioreactor Process

The batch process was initiated by inoculating N-1 seed in to a bioreactor containing growth medium at approx. 40±5% of the final batch volume at the starting VCC (after inoculation of the seed) in the monophasic Production bioreactor of 0.5±0.2×10⁶ cells/mL and viability >90%, more preferably VCC at 0.5×10⁶ cells/mL and viability >95% The agitation speed was set as per the tip speed ranging from 0.4 to 0.6 m/s. The temperature was set at a single set point i.e. 36° C. throughout the production bioreactor process. Feeding of the production bioreactor was done at day 2, 3, 5, 6, 7 and 8 for the cell culture maintenance, productivity and product quality attributes. Glutamine and Glucose were maintained throughout the cell culture duration at about 2 mM and 2 g/L respectively for cell culture maintenance. Harvesting of the culture was done on the ≦312 h or if the culture viability drops below 50%, whichever is earlier.

### Example 4

### Production of Adalimumab through Monophasic Process

Seed Expansion Process

A vial of Adalimumab from liquid nitrogen was thawed and the cells were inoculated in a 125 mL shake flask containing growth medium and was cultured in CO₂ incubator shaker at 37° C., 120 rpm. Cells were passaged every 72 h±24 h with increase in culture volume appropriate for seeding into production bioreactor. In each passage seeding density was maintained at 0.3×10⁶ cells/mL and target for a final VCC of about 3.0±1.0×10⁶ cells/mL.

Production Bioreactor Process

The batch process was initiated by inoculating N-1 seed in to a bioreactor containing growth medium at approx. 60±10% of the final batch volume at the starting VCC (after inoculation of the seed) in the monophasic Production bioreactor of 0.5±0.2×10⁶ cells/mL and viability >90%, more preferably VCC at 0.5×10⁶ cells/mL and viability >95%. The agitation speed was set as per the tip speed ranging from 0.6±0.2 m/s. The temperature was set at a single set point at 36° C. throughout the Production bioreactor process. Feeding was done on day 3, 6, 9 and 11 for the cell culture maintenance, productivity and product quality attributes. Glutamine and Glucose were maintained throughout the cell culture duration at about 2 mM and 2 g/L respectively for cell culture maintenance. The harvest criteria was set at ≦50% cell viability or 288 h±12 h whichever is earlier.

### Example 5

### Production of Trastuzumab through Monophasic Process

Seed Expansion Process

A vial of Trastuzumab from liquid nitrogen was thawed and the cells were inoculated in a 125 mL shake flask containing growth medium and was cultured in CO₂ incubator shaker at 37° C., 120 rpm, 5% CO₂, 85% Relative humidity. Cells were passaged every 72 h±24 h with increase in culture volume for inoculating production bioreactor. In each passage seeding density was maintained at 0.3×10⁶ cells/mL and target for a final VCC of about 3.0±1.0×10⁶ cells/mL.

Production Bioreactor Process

The batch process was initiated by inoculating N-1 seed in to a bioreactor containing growth medium at approx. 40±5% of the final batch volume at the starting VCC (after inoculation of the seed) in the monophasic production bioreactor of 0.5±0.2×10⁶ cells/mL and viability >90%, more preferably VCC at 0.5×10⁶ cells/mL and viability >95%. The agitation speed was set as per the tip speed ranging from 0.4 to 0.6 m/s. The temperature was at 34° C. throughout the production bioreactor process. Feeding of the reactor was done at day 2, 4, 6, 8 and 10 to maintain cell culture longevity, productivity and product quality attributes. Glutamine and Glucose were maintained throughout the cell culture duration at about 2 mM and 2 g/L respectively for cell culture maintenance. Harvesting of the culture was done on the ≦312 h or if the culture viability drops below 50%, whichever is earlier.

All patents, patent applications and publications cited in this application are hereby incorporated by reference in their entirety for all purposes to the same extent as if each individual patent, patent application or publication were so individually denoted.

1. A process of producing glycosylated protein in a mammalian cell culture the process comprising the steps of:
a) preparing inoculum with suitable cell concentration during seed development,
b) inoculating the inoculum with suitable cell concentration in to production bioreactor,
c) culturing the cell in production bioreactor at suitable conditions wherein the suitable condition is monophasic temperature condition; and
d) obtaining the glycosylated protein from the cell culture.

2. The process as claimed in claim 1, wherein monophasic temperature is selected from the range of about 32° C. to about 37° C.

3. The process as claimed in claim 1, wherein monophasic temperature is selected from the range of about 34° C. to about 37° C.

4. The process as claimed in claim 2, wherein monophasic temperature is about 33° C.

5. The process as claimed in claim 2, wherein monophasic temperature is about 34° C.

6. The process as claimed in claim 2, wherein monophasic temperature is about 35° C.

7. The process as claimed in claim 2, wherein monophasic temperature is about 36° C.

8. The process as claimed in claim 1, wherein monophasic temperature does not comprise temperature shift.

9. The process as claimed in claim 1, wherein the mammalian cell is selected from CHO cell.

10. The process as claimed in claim 9, wherein the CHO cell is a dhfr-CHO cell.

11. The process as claimed in claim 1, wherein the mammalian cells are cultured in fed-batch mode in production bioreactor.

12. The process as claimed in claim 1, wherein the cell culture process does not have distinctive growth phase and production phase.

13. The process as claimed in claim 1, wherein the suitable conditions further comprises pH selected from 6.7 to 7.4.

14. The process as claimed in claim 13, wherein the suitable conditions further comprises pH is about 7.

15. The process as claimed in claim 1, wherein the suitable conditions further comprises osmolality from about 250 to about 550 mOSm/Kg.

16. The process as claimed in claim 15, wherein the osmolality is about 270 mOSm/Kg.

17. The process as claimed in claim 1, wherein the mammalian cells are cultured in serum containing medium or serum free medium.

18. The process as claimed in claim 17, wherein the mammalian cells are cultured in serum free medium.

19. The process as claimed in claim 1, wherein the mammalian cells are cultured in medium essentially free of alkanoic acid or salt thereof.

20. The process as claimed in claim 19, wherein the alkanoic acid or salt thereof are selected from butyric acid, sodium butyrate or dibutyl cAMP.

21. The process as claimed in claim 1, wherein the suitable conditions further comprises dissolved oxygen concentration from about 30% to about 70%.

22. The process as claimed in claim 1, wherein the suitable concentration of inoculum is selected from about 4×10⁶ cells/mL to about 7×10⁶ cells/m L.

23. The process as claimed in claim 1, wherein the suitable concentration of inoculum is obtained at least by 72 hours.

24. The process as claimed in claim 1, wherein the protein is glycosylated protein which are selected from fusion proteins and monoclonal antibodies and fragment thereof.

25. The process as claimed in claim 24, wherein the fusion protein and monoclonal antibody and fragment thereof are selected from Abciximab; Abatacept; Adalimumab; Abrilumab; Afutuzumab; Aflibercept; Alemtuzumab; Alefacept; Alacizumab pegol; Anakinra; Arcitumomab; Atacicept; Atlizumab; Atorolimumab; Basiliximab; Baminercept; Bectumomab; Belimumab; Besilesomab; Bevacizumab; Biciromab; Belatacept; Brentuximab vedotin; Brodalumab; Canakinumab; Capromab pendetide; Catumaxomab; Certolizumab pegol; Cetuximab; Clivatuzumab tetraxetan; Daclizumab; Denosumab; Eculizumab; Edrecolomab; Efalizumab; Efungumab; Eloctate; Ertumaxomab; Etanercept; Etaracizumab; Fanolesomab; Farletuzumab; Fontolizumab; Gemtuzumab ozogamicin; Girentuximab; Golimumab; Ibritumomab tiuxetan; Igovomab; Imciromab; Infliximab; Ipilimumab; Labetuzumab; Mepolizumab; Motavizumab; Muromonab-CD3; Natalizumab; Nimotuzumab; Nofetumomab merpentan; Obinutuzumab; Ofatumumab; Omalizumab; Oregovomab; Palivizumab; Panitumumab; Pemtumomab; Pertuzumab; Ramucirumab; Ranibizumab; Raxibacumab; Rituximab; Rilonacept; Rovelizumab; Ruplizumab; Sulesomab; Tacatuzumab tetraxetan; Tefibazumab; Tocilizumab; Trastuzumab; Ado-Trastuzumab Emtansine; Tositumomab; TRBS07; Ustekinumab; Vedolizumab; Visilizumab; Votumumab; Zalutumumab; Zanolimumab.

26. The process as claimed in claim 24 wherein the fusion protein is Etanercept.

27. The process as claimed in claim 1, wherein the culture conditions maintains a high viable cells count.

28. The process as claimed in claim 27, wherein the viable cell count is selected from about 5×10⁶ to about 13×10⁶ cells/mL.

29. The process as claimed in claim 28, wherein the viable cell count is 11×10⁶ cells/m L.

30. The process as claimed in claim 1, wherein the process improves the desire confirmation of glycosylated proteins.

31. The process as claimed in claim 1, wherein the process improves the desire confirmation of TNFR-Fc fusion proteins such as Etanercept.

32. The process as claimed in claim 1, wherein the mammalian cells are cultured in production bioreactor for at least about 10 days to about 13 days.

33. The process as claimed in claim 32, wherein the mammalian cells are cultured in production bioreactor for at least 11 days.

34. A process of producing fusion protein and monoclonal antibody and fragment thereof in a mammalian cell culture the process comprising the steps of:
a) preparing inoculum with suitable cell concentration during seed development;
b) inoculating the inoculum with suitable cell concentration in to production bioreactor;
c) culturing the cell in production bioreactor at suitable conditions wherein the suitable condition is monophasic temperature condition; and
d) obtaining the glycosylated protein from the cell culture,
wherein the suitable conditions are
i) Monophasic temperature selected from about 34° C. to about 37° C.,
ii) pH is selected from 6.7 to 7.4,
iii) Osmolality from about 250 mOSm/Kg to about 550 mOSm/Kg.

35. A process of producing fusion protein and monoclonal antibody and fragment thereof in a mammalian cell culture the process comprising the steps of:
a) preparing inoculum with suitable cell concentration during seed development;
b) inoculating the inoculum with suitable cell concentration in to production bioreactor;
c) culturing the cell in production bioreactor at suitable conditions wherein the suitable condition is monophasic temperature condition; and
d) obtaining the glycosylated protein from the cell culture,
wherein the suitable conditions are
i) monophasic temperature selected from about 34° C. to about 37° C.,
ii) pH is selected from 6.7 to 7.4,
iii) osmolality from about 250 mOSm/Kg to about 550 mOSm/Kg,
wherein the medium essentially free of alkanoic acid or salt thereof; and
wherein the cell culture process does not have distinctive growth phase and production phase.

36. A process of producing TNFR-Fc fusion protein in a mammalian cell culture the process comprising the steps of:
a) preparing inoculum with suitable cell concentration during seed development;
b) inoculating the inoculum with suitable cell concentration in to production bioreactor;
c) culturing the cell in production bioreactor at suitable conditions wherein the suitable condition is monophasic temperature condition; and
d) obtaining the glycosylated protein from the cell culture,
wherein the suitable conditions are
i) monophasic temperature selected from at about 34° C. to about 37° C.,
ii) pH is selected from 6.7 to 7.4,
iii) osmolality from about 250 mOSm/Kg to about 550 mOSm/Kg.

37. The process as claimed in claim 1, wherein the cell is selected from CHO DUKX-B11, CHO S, CHO K1 or CHO DG44.

38. A fusion protein or monoclonal antibody and fragment thereof obtained from the process as claimed in claim 1.
